(19) United States
(12) Patent Application Publication
(10) Pub. No.: US 2013/0109720 A1
(43) Pub. Date: May 2, 2013

(54) INDOLE INHIBITORS OF CRAC

(71) Applicant: Hoffmann-La Roche Inc., Nutley, NJ (US)

(72) Inventors: Muzaffar Alam, Clifton, NJ (US); Daisy Joe DuBois, Menlo Park, CA (US); Ronald Charles Hawley, Glen Ridge, NJ (US); Joshua Kennedy-Smith, New York, NY (US); Ana Elena Minatti, Santa Monica, CA (US); Wylie Solang Palmer, Morristown, NJ (US); Tania Silva, New York, NY (US); Kshitij Chhabilbhai Thakkar, Clifton, NJ (US); Robert Stephen Wilhelm, Los Altos, CA (US)

(73) Assignee: Hoffmann-La Roche Inc., Nutley, NJ (US)

(21) Appl. No.: 13/656,830

(22) Filed: Oct. 22, 2012

Related U.S. Application Data
(60) Provisional application No. 61/554,196, filed on Nov. 1, 2011.

(51) Int. Cl. C07D 401/14; A61P 9/02; A61P 11/00; A61K 31/444

(52) U.S. Cl. 514/333; 546/256

(57) ABSTRACT

Disclosed are compounds of Formula (I):

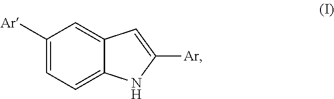

useful for treatment of autoimmune and inflammatory diseases associated with IL-2 inhibition via modulation of calcium release-activated calcium (CRAC) channels. Also disclosed are methods of making and using the compounds for treatment of diseases associated with CRAC channels.

### PRIORITY TO RELATED APPLICATION(S)

This application claims the benefit of U.S. Provisional Application No. 61/554,196, filed Nov. 1, 2011, which is hereby incorporated by reference in its entirety.

### RELATED APPLICATION(S)

This application is related to U.S. application Ser. No. 12/888,701, filed on Sep. 23, 2010, the entire contents of which are incorporated by reference herein.

### FIELD OF THE INVENTION

This invention pertains to compounds useful for treatment of autoimmune and inflammatory diseases associated with IL-2 inhibition via modulation of calcium release-activated calcium channels.

### BACKGROUND OF THE INVENTION

The cytokine interleukin 2 (IL-2) is a T-cell mitogen important for T-cell proliferation and as a B cell growth factor. Because of its effects on T cells and B cells, IL-2 is recognized as an important regulator of immune responses. IL-2 is involved in inflammation, tumor progression and hematopoiesis, and IL-2 affects the production of other cytokines such as TNA alpha, TNF beta, IFN gamma. Inhibition of IL-2 production thus is relevant to immunosuppression therapies and treatment of inflammatory and immune disorders.

T-cell antigen binding in inflammatory events leads to T-cell initiated calcium influx by calcium release-activated calcium channels (CRAC). IL-2 secretion by T-cells occurs in response to calcium ion influx. Modulation of CRAC thus provides a mechanism for control of production of IL-2 and other cytokines associated with inflammation. CRAC inhibition has been recognized as a potential route to therapies for rheumatoid arthritis, asthma, allergic reactions and other inflammatory conditions (see, e.g., Chang et al., Acta Pharmacologica Sinica (2006) Vol. 7, 813-820), and CRAC inhibitors have been shown to prevent antigen-induced airway eosinophilia and late phase asthmatic responses via Th2 cytokine inhibition in animal models (Yoshino et al., Eur. J. Pharm. (2007) Vol. 560(2), 225-233). There is, accordingly, a need for CRAC inhibitors.

### SUMMARY OF THE INVENTION

The invention provides a compound of Formula (I):

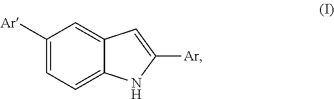

wherein:
Ar is pyridinyl, optionally substituted once or twice independently with lower alkyl or halogen; and
Ar′ is pyridinyl, optionally substituted once or twice independently with lower alkyl or —SO₂(CH₃),
or a pharmaceutically acceptable salt thereof.

The invention also provides for pharmaceutical compositions comprising the compounds, methods of using the compounds, and methods of preparing the compounds.

All documents cited to or relied upon below are expressly incorporated herein by reference.

### DETAILED DESCRIPTION OF THE INVENTION

### Definitions

Unless otherwise stated, the following terms used in this application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms “Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethyl ethylene, propylene, 2-methylpropylene, butylene, pentylene, and the like.

“Alkoxy” and “alkyloxy”, which may be used interchangeably, mean a moiety of the formula —OR, wherein R is an alkyl moiety as defined herein. Examples of alkoxy moieties include, but are not limited to, methoxy, ethoxy, isopropoxy, and the like.

“Amino means a moiety of the formula —NRR′ wherein R and R′ each independently is hydrogen or alkyl as defined herein. “Amino thus includes “alkylamino (where one of R and R′ is alkyl and the other is hydrogen) and “dialkylamino (where R and R′ are both alkyl.

“Aryl” means a monovalent cyclic aromatic hydrocarbon moiety having a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

“Cycloalkyl” means a monovalent saturated carbocyclic moiety having mono- or bicyclic rings. Preferred cycloalkyl are unsubstituted or substituted with alkyl. Cycloalkyl can optionally be substituted with one or more substituents, wherein each substituent is independently hydroxy, alkyl, alkoxy, halo, haloalkyl, amino, monoalkylamino, or dialkylamino, unless otherwise specifically indicated. Examples of cycloalkyl moieties include, but are not limited to, cyclopropyl, cyclobutyl, cyclopentyl, cyclohexyl, cycloheptyl, and the like, including partially unsaturated (cycloalkenyl) derivatives thereof

“Heteroalkyl” means an alkyl radical as defined herein wherein one, two or three hydrogen atoms have been replaced with a substituent independently selected from the group consisting of —OR^{a}, —NR^{b}R^{c}, and —S(O)ₙR^{d} (where n is an integer from 0 to 2), with the understanding that the point of attachment of the heteroalkyl radical is through a carbon atom, wherein R^{a} is hydrogen, acyl, alkyl, cycloalkyl, or cycloalkylalkyl; R^{b} and R^{c} are independently of each other hydrogen, acyl, alkyl, cycloalkyl, or cycloalkylalkyl; and when n is 0, R^{d} is hydrogen, alkyl, cycloalkyl, or cycloalkylalkyl, and when n is 1 or 2, R^{d} is alkyl, cycloalkyl, cycloalkylalkyl, amino, acylamino, monoalkylamino, or dialkylamino. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methyl sulfonylethyl, aminosulfonylmethyl, aminosulfonylethyl, aminosulfonylpropyl, methylaminosulfonylmethyl, methylaminosulfonylethyl, methylaminosulfonylpropyl, and the like.

“Heteroaryl” means a monocyclic or bicyclic radical of 5 to 12 ring atoms having at least one aromatic ring containing one, two, three or four ring heteroatoms selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, tetrazolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, including partially hydrogenated derivatives thereof, each optionally substituted.

The terms “halo”, “halogen” and “halide”, which may be used interchangeably, refer to a substituent fluoro, chloro, bromo, or iodo.

“Haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Heterocyclyl” means a monovalent saturated moiety, having one to three rings, incorporating one, two, or three or four heteroatoms (chosen from nitrogen, oxygen or sulfur). The heterocyclyl ring may be optionally substituted as defined herein. Examples of heterocyclyl moieties include, but are not limited to, optionally substituted piperidinyl, piperazinyl, homopiperazinyl, azepinyl, pyrrolidinyl, pyrazolidinyl, imidazolinyl, imidazolidinyl, pyridinyl, pyridazinyl, pyrimidinyl, oxazolidinyl, isoxazolidinyl, morpholinyl, thiazolidinyl, isothiazolidinyl, quinuclidinyl, quinolinyl, isoquinolinyl, benzimidazolyl, thiadiazolylidinyl, benzothiazolidinyl, benzoazolylidinyl, dihydrofuryl, tetrahydrofuryl, dihydropyranyl, tetrahydropyranyl, thiamorpholinyl, thiamorpholinylsulfoxide, thiamorpholinylsulfone, dihydroquinolinyl, dihydrisoquinolinyl, tetrahydroquinolinyl, tetrahydrisoquinolinyl, and the like.

“Urea” or “ureido” means a group of the formula —NR′—C(O)—NR″R′″ wherein R′, R″ and R′″ each independently is hydrogen or alkyl.

“Carbamate” means a group of the formula —O—C(O)—NR′R″ wherein R′ and R″ each independently is hydrogen or alkyl.

“Carboxy” means a group of the formula —O—C(O)—OH.

“Optionally substituted”, when used in association with “aryl”, phenyl”, “heteroaryl” “cycloalkyl” or “heterocyclyl”, means an aryl, phenyl, heteroaryl, cycloalkyl or heterocyclyl which is optionally substituted independently with one to four substituents, preferably one or two substituents selected from alkyl, cycloalkyl, cycloalkylalkyl, heteroalkyl, hydroxyalkyl, halo, nitro, cyano, hydroxy, alkoxy, amino, acylamino, mono-alkylamino, haloalkyl, haloalkoxy, heteroalkyl, —COR, —SO₂R (where R is hydrogen, alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl), or (CR′R″)ₙ—CONR^{a}R^{b} (where n is an integer from 0 to 5, R′ and R″ are independently hydrogen or alkyl, and R^{a} and R^{b} are, independently of each other, hydrogen, alkyl, cycloalkyl, cycloalkylalkyl, phenyl or phenylalkyl). Certain preferred optional substituents for “aryl”, phenyl”, “heteroaryl” “cycloalkyl” or “heterocyclyl” include alkyl, halo, haloalkyl, alkoxy, cyano, amino and alkylsulfonyl. More preferred substituents are methyl, fluoro, chloro, trifluoromethyl, methoxy, amino and methanesulfonyl.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:

acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or
salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition salt.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate.

Hydrates are formed by the combination of one or more molecules of water with one of the substances in which the water retains its molecular state as H₂O, such combination being able to form one or more hydrate.

“Subject” means mammals and non-mammals. Mammals means any member of the mammalian class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Arthritis” means diseases or conditions damage to joints of the body and pain associated with such joint damage. Arthritis includes rheumatoid arthritis, osteoarthritis, psoriatic arthritis, septic arthritis and gouty arthritis.

“Pain” includes, without limitation, inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes:

preventing the disease state, i.e. causing the clinical symptoms of the disease state not to develop in a subject that may be exposed to or predisposed to the disease state, but does not yet experience or display symptoms of the disease state:
inhibiting the disease state, i.e., arresting the development of the disease state or its clinical symptoms, or
relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

In general, the nomenclature used in this application is based on AUTONOM™ v.4.0, a Beilstein Institute computerized system for the generation of IUPAC systematic nomenclature. Chemical structures shown herein were prepared using ISIS® version 2.2. Any open valency appearing on a carbon, oxygen sulfur or nitrogen atom in the structures herein indicates the presence of a hydrogen atom unless indicated otherwise. Where a nitrogen-containing heteroaryl ring is shown with an open valency on a nitrogen atom, and variables such as R^{a}, R^{b} or R^{c} are shown on the heteroaryl ring, such variables may be bound or joined to the open valency nitrogen. Where a chiral center exists in a structure but no specific stereochemistry is shown for the chiral center, both enantiomers associated with the chiral center are encompassed by the structure. Where a structure shown herein may exist in multiple tautomeric forms, all such tautomers are encompassed by the structure. The atoms represented in the structures herein are intended to encompass all naturally occurring isotopes of such atoms. Thus, for example, the hydrogen atoms represented herein are meant to include deuterium and tritium, and the carbon atoms are meant to include C¹³ and C¹⁴ isotopes.

In one embodiment, the invention provides for a compound of Formula (I):

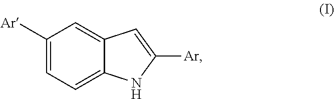

wherein:
Ar is pyridinyl, optionally substituted once or twice independently with lower alkyl or halogen; and
Ar′ is pyridinyl, optionally substituted once or twice independently with lower alkyl or —SO₂(CH₃),
or a pharmaceutically acceptable salt thereof.

In another embodiment, the invention provides for a compound according to Formula (I), wherein the lower alkyl in each instance is methyl, ethyl, propyl, isopropyl, isobutyl, sec-butyl or tert-butyl.

In another embodiment, the invention provides for a compound according to Formula (I), wherein halogen is fluorine or chlorine.

In another embodiment, the invention provides for a compound according to Formula (I), wherein Ar is pyridinyl bi-substituted with methyl and halogen.

In another embodiment, the invention provides for a compound according to Formula. (I), wherein Ar′ is pyridinyl bi-substituted with methyl and —SO₂(CH₃).

In another embodiment, the invention provides for a compound according to Formula (I), wherein the compound is 2-(5-fluoro-4-methyl-pyridin-3-yl)-5-(6-methanesulfonyl-4-methyl-pyridin-3-yl)-1H-indole.

In another embodiment, the invention provides for a pharmaceutical composition, comprising a therapeutically effective amount of a compound according to Formula (I) and a pharmaceutically acceptable carrier.

In another embodiment, the invention provides for a compound according to Formula (I) for use as a therapeutically active substance.

In another embodiment, the invention provides for a use of a compound according to Formula (I) for the treatment or prophylaxis of arthritis or a respiratory disorder.

In another embodiment, the invention provides for a use of a compound according to Formula (I) for the preparation of a medicament for the treatment or prophylaxis of arthritis or a respiratory disorder.

In another embodiment, the invention provides for a compound according to Formula (I) for the treatment or prophylaxis of arthritis or a respiratory disorder.

In another embodiment, the invention provides for a method for treating arthritis, comprising the step of administering a therapeutically effective amount of a compound according to Formula (I) to a subject in need thereof.

In another embodiment, the invention provides for a method for treating a respiratory disorder selected from chronic obstructive pulmonary disorder (COPD), asthma, and bronchospasm, comprising the step of administering a therapeutically effective amount of a compound according to Formula (I) to a subject in need thereof.

In a further embodiment, provided is an invention as hereinbefore described.

The invention also provides methods for treating a disease or condition mediated by or otherwise associated with a CRAC receptor, the method comprising administering to a subject in need thereof an effective amount of a compound of the invention.

The invention also provides methods for treating an inflammatory, respiratory or diabetes condition, the method comprising administering to a subject in need thereof an effective amount of a compound of the invention together with an effective amount of a CRAC inhibitor.

The disease may be an inflammatory disease such as arthritis, and more particularly rheumatoid arthritis, osteoarthritis, psoriasis, allergic dermatitis, asthma, chronic obstructive pulmonary disease, airways hyper-responsiveness, septic shock, glomerulonephritis, irritable bowel disease, and Crohn's disease.

The disease may be a pain condition, such as inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome,

The disease may be a respiratory disorder, such as chronic obstructive pulmonary disorder (COPD), asthma, or bronchospasm, or a gastrointestinal (GI) disorder such as Irritable Bowel Syndrome (IBS), Inflammatory Bowel Disease (IBD), biliary colic and other biliary disorders, renal colic, diarrhea-dominant IBS, pain associated with GI distension.

### Synthesis

Compounds of the present invention can be made by a variety of methods depicted in the illustrative synthetic reaction schemes shown and described below.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

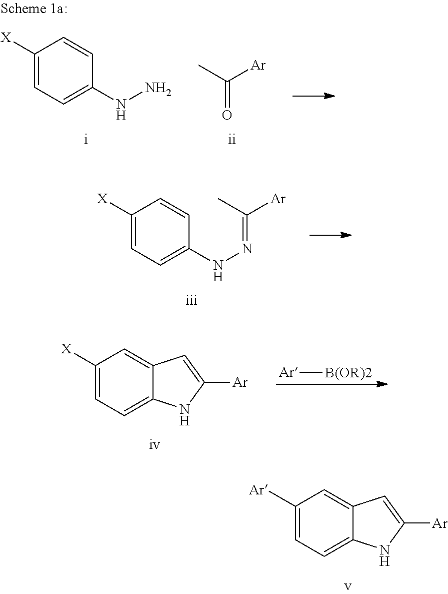

As shown in Scheme 1a, an aryl hydrazine i, where X=halide, can be reached with an appropriate acetophenone ii, to give hydrazone iii. The hydrazone iii can then be reacted in the presence of polyphosphoric acid under Fischer indole synthesis conditions to give a 2-aryl-5-halo-indole iv. Suzuki coupling of indole iv with an appropriate boronic acid or ester then gives 2,5-diaryl-indole v. Ar and Ar′, for example, can be, independently of each other, an unsubstituted heteroaryl group or a mono- or bi-substituted heteroaryl group.

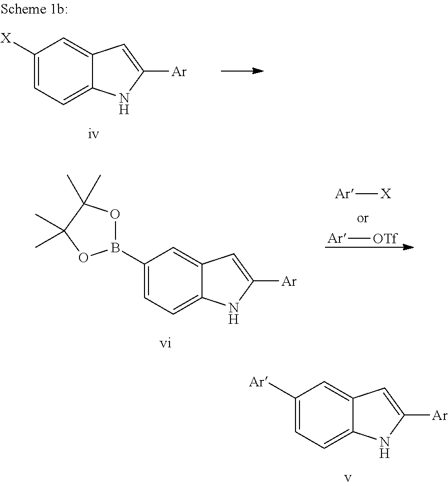

As shown in Scheme 1b, 2-aryl-5-halo-indole iv, can also be converted to the indole-boronic ester vi in the presence of a palladium catalyst and bispinacolatodiborane. Suzuki coupling of indole-boronic ester vi with an appropriate aryl halide or triflate then gives 2,5-diaryl-indole v. Ar and Ar′ are as defined above in Scheme 1a.

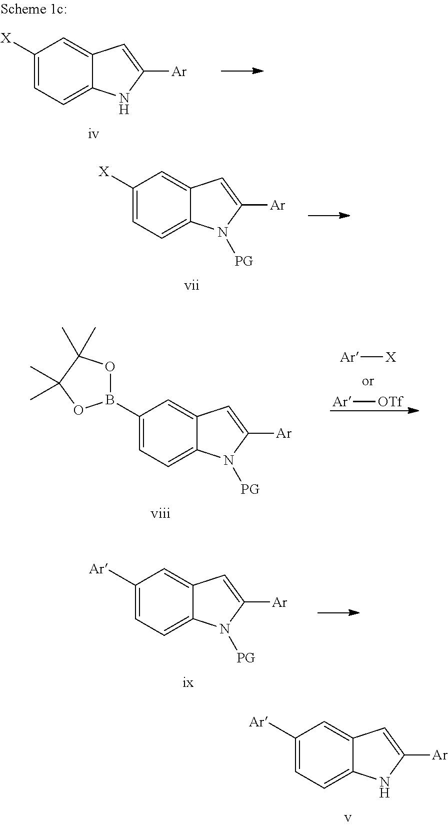

As shown in Scheme 1c, the indole functionality in 2-aryl-5-halo-indole iv can be protected to give protected indole vii. Indole vii can then be converted to the protected indole-boronic ester viii in the presence of a palladium catalyst and bispinacolatodiborane. Suzuki coupling of indole viii with an appropriate aryl halide or triflate then gives protected 2,5-diaryl-indole ix. This indole ix can be deprotected under basic conditions to give 2,5-diaryl-indole v. Ar and Ar′ are as defined above in Scheme 1a.

Many variations on the procedure of the above Schemes are possible and will suggest themselves to those skilled in the art. Specific details for producing compounds of the invention are described in the Examples section below.

### Utility

The compounds of the invention are usable for the treatment of a wide range of inflammatory diseases and conditions such as arthritis, including but not limited to, rheumatoid arthritis, spondyloarthropathies, gouty arthritis, osteoarthritis, systemic lupus erythematosus and juvenile arthritis, osteoarthritis, gouty arthritis and other arthritic conditions. The subject compounds would be useful for the treatment of pulmonary disorders or lung inflammation, including adult respiratory distress syndrome, pulmonary sarcoidosis, asthma, silicosis, and chronic pulmonary inflammatory disease.

Further, compounds of the invention are useful for treating respiratory disorders, including chronic obstructive pulmonary disorder (COPD), asthma, bronchospasm, and the like.

### Administration and Pharmaceutical Composition

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention will be administered in a therapeutically effective amount by any of the accepted modes of administration for agents that serve similar utilities. Suitable dosage ranges are typically 1-500 mg daily, preferably 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compounds of the present invention for a given disease.

Compounds of the invention may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention may be formulated for vaginal administration. Pessaries, tampons, creams, gels, pastes, foams or sprays containing in addition to the active ingredient such carriers as are known in the art to be appropriate.

The subject compounds may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form

Other suitable pharmaceutical carriers and their formulations are described in Remington. The Science and Practice of Pharmacy 1995, edited by E. W. Martin, Mack Publishing Company, 19th edition, Easton, Pa. Representative pharmaceutical formulations containing a compound of the present invention are described below.

### EXAMPLES

The following preparations and examples are given to enable those skilled in the art to more clearly understand and to practice the present invention. They should not be considered as limiting the scope of the invention, but merely as being illustrative and representative thereof.

Unless otherwise stated, all temperatures including melting points (i.e., MP) are in degrees celsius (° C.). It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The following abbreviations may be used in the Preparations and Examples:

### DMF N,N-dimethylformamide

DMSO dimethyl sulfoxide
EtOAc ethyl acetate
hplc high performance liquid chromatography
MeOH methanol
THF tetrahydrofuran

### Part I: Preparation of Certain Intermediates Preparation of 5-bromo-2-(5-fluoro-4-methyl-pyridin-3-yl)-1H-indole

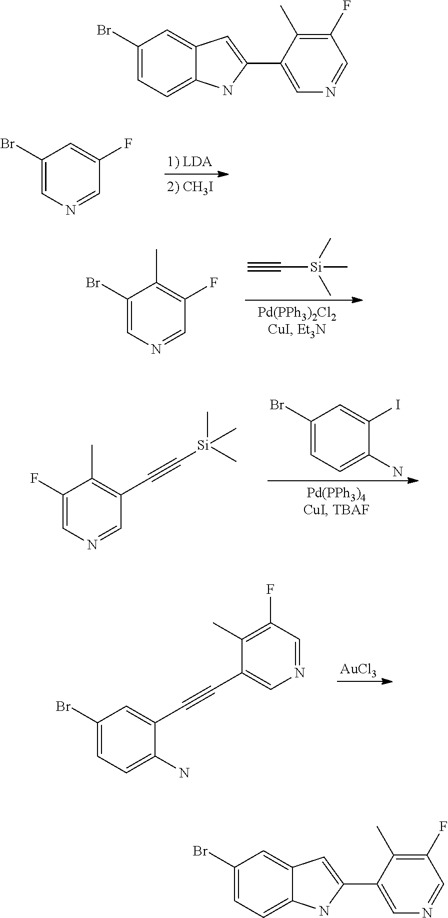

### Step 1:

In a 250 mL round-bottomed flask, LDA, 2.0M in heptane/THF/ethylbenzene (9.4 ml, 18.8 mmol, Eq: 1.10) was combined with THF (15 ml) to give an orange solution. The mixture was cooled to −78° C. and 3-bromo-5-fluoropyridine (3 g, 17.0 mmol, Eq: 1.00) in THF (15 ml) was added dropwise over 10 min. The brown reaction mixture was stirred at −78° C. for 30 min. A solution of iodomethane (3.63 g, 1.6 ml, 25.6 mmol, Eq: 1.5) in THF (15.0 ml) was added dropwise over 5 min. The mixture was stirred at −78° C. for 30 minutes. Cooling was removed and stirring continued for 1 h. aq. A saturated ammonium chloride solution was added cautiously followed by ethyl acetate. The ethyl acetate layer was separated and the aqueous layer was extracted 2× with ethyl acetate. The organic layers were combined, washed with aq, sodium thiosulfate, washed with brine, dried over anhydrous sodium sulfate, filtered and concentrated. The crude material was purified by flash chromatography (silica gel, 120 g, 0% to 5% EtOAc in hexanes). Fraction were pooled and evaporated to yield 1.69 g (52%) of 3-bromo-5-fluoro-4-methylpyridine as a colorless oil. ¹H NMR (CHLOROFORM-d) δ: 8.53 (s, 1H), 8.35 (s, 1H), 2.41 (d, J=2.1 Hz, 3H). LC-MS (ES) calculated for C₆H₅BrFN, 190.02. found m/z 189.7 [M+H]⁺.

### Step 2:

3-Bromo-5-fluoro-4-methylpyridine (1.69 g, 8.89 mmol, Eq: 1.00), bis(triphenylphosphine)-palladium(10 dichloride (312 mg, 445 μmol, Eq: 0.05) and copper (I) iodide (84.7 mg, 445 μmol, Eq: 0.05) in DMF (15 ml) with flushed with nitrogen and treated with ethynyltrimethylsilane (1.05 g, 1.5 ml, 10.7 mmol, Eq: 1.2) and triethylamine (3.63 g, 5 ml, 35.9 mmol, Eq: 4.03). The reaction was heated to 115° C. and held at this temperature for 18 h.

The mixture was cooled, diluted with water and extracted with ether (3×). The combined organic layers were washed with water (2×), brine, dried over anhydrous sodium sulfate, filtered through celite and concentrated to give a brown oil. The crude material was purified by flash chromatography (silica gel, 80 g, 20% EtOAc in hexanes). Fraction were pooled and evaporated to yield 1.09 g (59%) of 3-fluoro-4-methyl-5-((trimethylsilyl)ethynyl)pyridine as a yellow oil containing some solid. ¹H NMR (DMSO-d₆) δ 8.50 (s, 1H), 8.44 (s, 1H), 2.34 (d, J=1.8 Hz, 3H), 0.27 (s, 9H). LC-MS (ES) calculated for C₁₁H₁₄FNSi, 207.33. found m/z 207.8 [M+H]⁺.

### Step 3:

In a 100 mL round bottom flask with a reflux condenser under argon, 3-fluoro-4-methyl-5-(trimethylsilyl)ethynyl)pyridine (2.84 g, 10.8 mmol, Eq: 1.00), 4-bromo-2-iodoaniline (3.22 g, 10.8 mmol, Eq: 1.00)₂ tetrakis(triphenylphosphine)palladium(0) (625 mg, 541 μmol, Eq: 0.05), copper(I) iodide (105 mg, 551 mmol, Eq: 0.0509), tetrabutylammonium fluoride (0.00 μg, 11 mL, 11.0 mmol, Eq: 1.02), and triethylamine (3.82 g, 5.25 ml, 37.8 mmol, Eq: 3.49) were all combined in THF (50 ml) and vacuum flushed with argon. The reaction was stirred at 65° C. for 1 h. The reaction was concentrated in vacuo and dissolved in EtOAc/MeOH (9:1, 100 mL). The solution was washed with water (75 mL), 5% NaHCO₃ solution (75 mL), water (50 mL), and brine (50 mL). The organic phases were dried over Na₂SO₄ and concentrated. The grey solid residue was dissolved in CH₂Cl₂ (50 mL) and purified by flash chromatography (silica gel, 220 g, 40% EtOAc in hexanes). Fraction containing product were pooled and evaporated to yield 4-bronco-2-((5-fluoro-4-methylpyridin-3-yl)ethynyl)aniline as a yellow solid, (2.048 g, 62.0%). ¹H NMR (DMSO-d₆) δ: 8.66 (br. s., 1H), 8.48 (br. s., 1H), 7.45 (br. s., 1H), 7.26 (d, J=8.5 Hz, 1H), 6.72 (d, J=8.8 Hz, 1H), 5.83 (br. s., 2H), 2.42 (hr. s., 3H). LC-MS (ES) calculated for C₁₄H₁₀BrFN₄, 305.15. found m/z 304.9 [M+H]⁺.

### Step 4:

In a 250 mL round-bottomed flask, 4-bromo-2-((5-fluoro-4-methylpyridin-3-yl)ethynyl)aniline (2.447 g, 8.02 mmol, Eq: 1.00) and gold(III) chloride (150 mg, 495 μmol, Eq: 0.0617) were dissolved in ethanol (100 ml) and heated at 65° C. for 18 h. The reaction was cooled, diluted with ethyl acetate (400 mL), filtered through a plug of Fluorsil and Celite and concentrated. The pad was washed with ethyl acetate (100 mL) and the combined filtrates evaporated to yield 2.50 g (102%) of a brown solid. The solid was dissolved in a minimum of hot EtOAc (30 mL). The product was precipitated by dropwise addition of hexanes (50 mL) at rt. The product was isolated by filtration and dried in vacuo (40° C., 100 mTorr) to give a brown solid, 1.232 g. The mother liquor was concentrated and redissolved in hot EtOAc (10 mL). The product was precipitated by dropwise addition of hexanes (50 mL) and isolated by filtration and dried in vacuo (40° C., 100 mTorr) to give a brown solid, 0.745 g. Crops were combined to give 5-bromo-2-(5-fluoro-4-methylpyridin-3-yl)-1H-indole (1.977 g, 81% yield) and used as is for next reaction. ¹H NMR (DMSO-d₆) δ: 11.78 (s, 1H), 8.60 (s, 1H), 8.53 (s, 1H), 7.80 (s, 1H), 7.41 (d, J=8.8 Hz, 1H), 7.28 (d, J=8.5 Hz, 1H), 6.75 (s, 1H), 2.42 (s, 3H), LC-MS (ES) calculated for C₁₄H₁₀BrFN₄, 305.15. found m/z: 304.9 [M+H]⁺.

### Preparation of 5-bromo-2-methanesulfonyl-4-methyl-pyridine

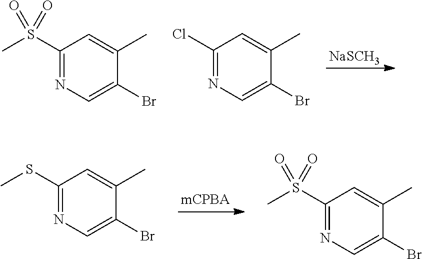

### Step 1:

In a 250 mL round-bottomed flask, 5-bromo-2-chloro-4-methylpyridine (14.8 g, 71.7 mmol, Eq: 1.00) and sodium thiomethoxide (7.03 g, 100 mmol, Eq: 1.40) were combined with dioxane (65 ml) to give a light yellow suspension. The mixture was heated to reflux for 22.5 h. The mixture was cooled, diluted with 250 mL EtOAc, washed with 2×100 mL water, 1×75 mL sat NaCl, dried over MgSO₄, filtered and concentrated to a light yellow oil (17 g). This material was used without further purification. LC-MS (ES) calculated for C₇H₈BrNS, 218.12. found m/z 218.0 [M+H]⁺.

### Step 2:

In a 250 mL round-bottomed flask, 5-bromo-4-methyl-2-(methylthio)pyridine (10.46 g, 39.3 mmol, Eq: 1.00) was combined with dichloromethane (160 ml) to give a colorless solution. The solution was chilled in an ice bath and 3-chloroperoxybenzoic acid (19.0 g, 62.9 mmol, Eq: 1.60) was added in small (˜1-2 g) portions. LC-MS at t=1.5 h showed the reaction was complete. The reaction mixture was poured into 200 mL CH₂Cl₂, washed with 2×100 mL sat NaHCO₃ (diluted by 50% with water), and 75 mL sat NaCl, dried over MgSO₄, filtered and evaporated to an oily solid (18 g). The crude material (adsorbed onto silica) was purified by flash chromatography (silica gel, 40×240 g, 20% to 50% EtOAc in hexanes). Fractions containing product were pooled and evaporated to give 5-bromo-2-methanesulfonyl-4-methyl-pyridine as a white solid (5.63 g, 57%). ¹H NMR (CHLOROFORM-d) δ: 8.74 (s, 1H), 7.96 (s, 1H), 3.22 (s, 3H), 2.53 (s, 3H). LC-MS (ES) calculated for C₇H₈BrNO2S, 250.12. found m/z 251.9 [M+H]⁺.

### Part II: Preparation of Certain Embodiments of the Invention

### Example 1

### 2-(5-fluoro-4-methyl-pyridin-3-yl)-5-(6-methanesulfonyl-4-methyl-pyridin-3-yl)-1H-indole

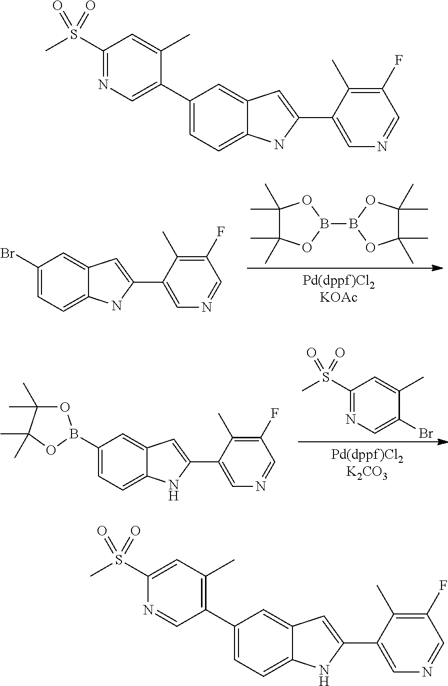

### Step 1:

In a 250 mL round bottom flask under argon, 5-bromo-2-(5-fluoro-4-methylpyridin-3-yl)-1H-indole (4.40 g, 14.4 mmol, Eq: 1.00), 4,4,4′,4′,5,5,5′,5′-octamethyl-2,2′-bi(1,3,2-dioxaborolane) (4.40 g, 17.3 mmol, Eq: 1.2), [1,1-bis(diphenylphosphino)ferrocene]dichloropalladium(II) (1.10 g, 1.5 mmol, Eq: 0.104), and potassium acetate (4.25 g, 43.3 mmol, Eq: 3) were combined in dioxane (100 ml) and stirred at 100° C. for 3 hours. The reaction was cooled to room temperature and filtered through Celite and Fluorsil. The plug was washed with EtOAc (500 mL). The combined filtrates were washed with water (2×250 mL) and saturated NaCl (1×250 mL) and dried over Na₂SO₄. The solvent was removed and residue was dried in vacuo to give 2-(5-fluoro-4-methylpyridin-3-yl)-5-(4,4,5,5-tetramethyl-1,3,2-dioxaborolan-2-yl)-1H-indole as a brown oil (7.88 g, 155%). The product was carried forward without further purification. LC-MS (ES) calculated for C₂₀H₂₂BFN₂O₂, 352.22. found m/z 353.0 [M+H]⁺.

### Step 2:

In a 25 mL 3-neck round bottom flask, with a reflux condenser, 2-(5-fluoro-4-methylpyridin-3-yl)-5-(4,4,5,5-tetramethyl-1,3,2-dioxaborolan-2-yl)-1H-indole (5.11 g, 14.5 mmol, Eq: 1.05), 5-bromo-4-methyl-2-(methylsulfonyl)pyridine (3.4551 g, 13.8 mmol, Eq: 1.00), [1,1′-bis(diphenylphosphino)ferrocene]dichloropalladium(II) (1.01 g, 1.38 mmol, Eq: (0.100), and potassium carbonate (5.75 g, 41.6 mmol, Eq: 3.01) were dissolved in dioxane (200 mL) and water (40 mL), vacuum purged with Ar (3×), and heated to 100° C. for 1 hour. The reaction was cooled to room temperature and diluted with EtOAc (250 mL). The organics were washed with water 250 mL, filtered through Fluorosil and Celite, re-washed with water (250 mL), saturated NaCl (250 mL) and dried over Na₂SO₄. The solvent was removed to yield a brown foam. The crude product was purification by column chromatography (silica gel, 50-75% gradient of ethyl acetate in hexane). Fractions containing product were pooled and evaporated to an off-white foam and was triturated with hexanes. The solid was isolated by filtration and dried in vacuo (40° C., 200 mTorr) to give 2-(5-fluoro-4-methylpyridin-3-yl)-5-(4-methyl-6-(methylsulfonyl)pyridin-3-yl)-1H-indole as an off-white foam (3.854 g, 70.6%). ¹H NMR (DMSO-d₆) δ: 11.78 (s, 1H), 8.62 (s, 2H), 8.53 (s, 1H), 8.05 (s, 1H), 7.70 (d, 1H), 7.58 (d, J=8 Hz, 1H), 7.24 (dd, J=8, 1 Hz, 1H), 6.82 (s, 1H), 3.30 (s, 3H), 2.48 (d, J=2.1 Hz, 3H) 2.43 (s, 3H). LC-MS (ES) calculated for C₂₁H₁₈FN₃O₂S, 395.46. found 117/Z 396.0 [M+H]⁺.

### Example 2

### Jurkat IL-2 Production Assay

Cell: Jurkat cell (ATCC) was grown in RPMI 1640 with 10% FBS and 1% penicillin/streptomycin. The cell density was kept at 1.2˜1.8×10⁶/mL in culture flask before seeding into culture plate, and the cell density in the plate was 0.5×10⁶/200 μL/well.

Culture media: RPMI 1640 with 1% FBS or 30% FBS for high serum assay.

Test compound: serial dilution was done in 100% DMSO, and intermediate dilution was done with RPMI 1640 medium with 1% FBS. The DMSO final concentration in culture well was 0.25%.

Stimulant: PHA (Sigma#L9017-10MG) was used for the assay with 1% FBS in culture medium, and added after 10 minutes exposure of cell to compound/DMSO. The PT-1A final concentration in culture well was 5 μg/mL. PMA (Sigma# P-8139 5MG)/Ionomycin (Sigma# I0634-5MG) was used for the assay with 30% FBS in culture medium, and added at same time point as the 1% FBS culture assay. The final concentration of PMA was 50 ng/mL, and Ionomycin final concentration was 500 ng/mL.

Incubation: at 37° C. with 5% CO₂ and 95% humidity for 18 h ˜20 h.

IC50: IC50 was calculated with the data analysis software XLfit4, General Pharmacology model 251.

Using the above procedure, the IC₅₀ value for the compound of Example 1 was 29 nM.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

1. A compound of Formula (I): 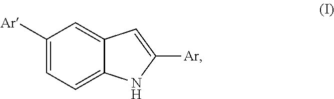
wherein
Ar is pyridinyl, optionally substituted once or twice independently with lower alkyl or halogen; and
Ar′ is pyridinyl, optionally substituted once or twice independently with lower alkyl or —SO₂(CH₃),
or a pharmaceutically acceptable salt thereof.

2. The compound according to claim 1, wherein said lower alkyl in each instance is methyl, ethyl, propyl, isopropyl, isobutyl, sec-butyl or tert-butyl.

3. The compound according to claim 1, wherein said halogen is fluorine or chlorine.

4. The compound according to claim 1, wherein Ar is pyridinyl bi-substituted with methyl and halogen.

5. The compound according to claim 1, wherein Ar′ is pyridinyl bi-substituted with methyl and —SO₂(CH₃).

6. The compound according to claim 1, wherein said compound is 2-(5-fluoro-4-methyl-pyridin-3-yl)-5-(6-methanesulfonyl-4-methyl-pyridin-3-yl)-1H-indole.

7. A pharmaceutical composition, comprising a therapeutically effective amount of a compound according to claim 1 and a pharmaceutically acceptable carrier.

8. A method for treating arthritis or a respiratory disorder, comprising the step of administering a therapeutically effective amount of a compound according to claim 1 to a subject in need thereof.
